(19) United States
(12) Patent Application Publication
(10) Pub. No.: US 2008/0140082 A1
(43) Pub. Date: Jun. 12, 2008

(54) KIT AND METHODS FOR MEDICAL PROCEDURES WITHIN A SACRUM

(76) Inventors: Eren Erdem, Little Rock, AR (US); Lisanne A. Eng, Campbell, CA (US); Christopher U. Phan, San Leandro, CA (US); Meera Sankaran, Cupertino, CA (US); Ed Asturias, San Francisco, CA (US)

Correspondence Address: COOLEY GODWARD KRONISH LLP;ATTN: PATENT GROUP, Suite 1100, 777 - 6th Street, NW, WASHINGTON, DC, 20001, US

(21) Appl. No.: 11/778,453

(22) Filed: Jul. 16, 2007

Related U.S. Application Data
(60) Provisional application No. 60/831,259, filed on Jul. 17, 2006.

(51) Int. Cl. A61B 17/56

(52) U.S. Cl. 606/92; 206/572

(57) ABSTRACT

Devices and methods for performing a procedure within a sacrum are disclosed herein. In one variation, a method includes imaging a spine with a fluoroscopy device to provide a view of the sacrum. An anatomical landmark is identified based on the imaging, and a breach zone is defined based on the imaging. The anatomical landmark is used to identify an entry point and guide a medical device in a medial-to-lateral approach into a sacral ala region of the sacrum to perform a medical procedure within the sacral ala. In some embodiments, the anatomical landmark can be, for example, a pedicle, and in some embodiments, the anatomical landmark can be, for example, a V notch. In some embodiments, an entry point is identified using two anatomical landmarks. For example, the anatomical landmarks can be an S1 foramen of the side being accessed and a sacroiliac joint.

### CROSS-REFERENCE TO RELATED APPLICATIONS

This application claims priority to U.S. Provisional Patent Application Ser. No. 60/831,259, entitled “Methods For Medical Procedures Within A Sacrum,” filed Jul. 17, 2006, the disclosure of which is hereby incorporated by reference in its entirety.

### BACKGROUND

The invention relates generally to medical devices and procedures, including, for example, medical devices and methods for percutaneous treatment of a sacrum.

Known medical devices are configured to access percutaneously a vertebra or other area of a spine to perform a variety of different medical procedures. Other known devices and procedures are used to treat sacral insufficiency fractures within a sacrum of a spine. Such procedures can include the injection of a bone cement into the sacral ala region of a sacrum. The placement of medical instruments in the sacral ala region can be difficult due to poor fluoroscopy imaging procedures. In many cases, computed tomography (CT) guidance is needed to place a tool in the targeted area. Often, the physician performing the procedure does not have access to CT imaging equipment and, therefore this type of procedure cannot be performed.

Another problem can exist during the delivery of bone cement or other material to the targeted region of the sacral ala. Typically such materials travel to the path of least resistance, which can create a “spider web” effect on delivery. For example, the material is scattered and less concentrated in the targeted area.

Thus, a need exists for a kit and methods for performing procedures within a sacrum that can provide proper access to a targeted region within the sacrum, and provide controlled delivery of a bone filler material to the targeted region.

### SUMMARY OF THE INVENTION

Devices and methods for performing procedures within a sacrum of a spine are disclosed herein. In one embodiment, a method includes imaging a spine with a fluoroscopy device to provide a view of the sacrum. An anatomical landmark is identified, and a breach zone is defined based on the imaging. The anatomical landmark is used to identify an entry point and guide a medical device in a medial-to-lateral approach into a sacral ala region of the sacrum to perform a medical procedure within the sacral ala. In some embodiments, the anatomical landmark can be, for example, a pedicle, and in some embodiments, the anatomical landmark can be, for example, a V notch of a sacrum. In some embodiments, an entry point is identified using two anatomical landmarks. For example, the anatomical landmarks can be an S1 foramen of the side being accessed and a sacroiliac joint.

### BRIEF DESCRIPTION OF THE DRAWINGS

FIG. 1 is a rear or posterior view of a dorsal surface of a sacrum of a spine.

FIG. 2 is a front or anterior view of a ventral surface of the sacrum of FIG. 1.

FIG. 3 is a side or lateral view of a lateral surface of the sacrum of FIGS. 1 and 2.

FIG. 4 is a posterior view of a sacrum illustrating a sacral fracture.

FIG. 5 is a schematic illustration of medical devices used to perform methods according to embodiments of the invention.

FIG. 6 is a lateral view of a sacrum.

FIG. 7 is a reproduction of a fluoroscopic image of a lateral view of a sacrum.

FIG. 8 is a reproduction of a fluoroscopic image of an anteroposterior view of a sacrum.

FIG. 9 is a side view of C-arm device.

FIG. 10 is a reproduction of a fluoroscopic image of an anteroposterior view of the sacrum of FIG. 8.

FIG. 11 is a reproduction of a fluoroscopic image of a lateral view of the sacrum of FIG. 8.

FIG. 12 is a reproduction of a fluoroscopic image of a lateral view of the sacrum of FIG. 8 showing a medical device inserted at least partially within the sacrum.

FIG. 13 is a reproduction of a fluoroscopic image of a lateral view of the sacrum of FIG. 8 showing a breach zone.

FIG. 14 is a reproduction of a fluoroscopic image of a lateral view of the sacrum of FIG. 8 showing a medical device disposed partially therein.

FIG. 15 is a reproduction of a fluoroscopic image of a lateral view of the sacrum of FIG. 8 showing a medical device disposed partially therein.

FIG. 16 is a reproduction of a fluoroscopic image of a lateral view of the sacrum of FIG. 8 showing a medical device disposed partially therein.

FIG. 17 is a reproduction of a fluoroscopic image of an anteroposterior view of the sacrum of FIG. 8 showing a medical device disposed partially therein.

FIG. 18 is a reproduction of a fluoroscopic image of a sacroiliac view of the sacrum of FIG. 8.

FIG. 19 is a reproduction of a fluoroscopic image of an anteroposterior view of the sacrum of FIG. 8 illustrating bone cement disposed within the sacral ala.

FIG. 20 is a reproduction of a fluoroscopic image of a lateral view of the sacrum of FIG. 8 illustrating bone cement disposed within the sacral ala.

FIGS. 21-22 are reproductions of fluoroscopic images of an anteroposterior view of a sacrum.

FIGS. 23 and 24 are reproductions of fluoroscopic images of a lateral view of the sacrum of FIGS. 21 and 22 showing various medical devices disposed partially therein.

FIG. 25 is a reproduction of a fluoroscopic image of an oblique view of the sacrum of FIGS. 21-24.

FIG. 26 is a reproduction of a fluoroscopic image of a lateral view of the sacrum of FIGS. 21-25.

FIGS. 27 and 28 are each a different reproduction of a fluoroscopic image of a lateral view of the sacrum of FIGS. 21-26.

FIG. 29 is a reproduction of a fluoroscopic image of an anteroposterior view of the sacrum of FIGS. 21 and 22 showing a medical device disposed partially therein.

FIG. 30 is a reproduction of a fluoroscopic image of a sacroiliac view of the sacrum of FIGS. 21 and 22 showing bone cement disposed within the sacral ala regions.

FIG. 31 is a reproduction of a fluoroscopic image of a lateral view of the sacrum of FIGS. 21 and 22 showing bone cement disposed within the sacral ala region.

FIG. 32 is a rear or posterior view of a sacrum according to an embodiment of the invention.

FIG. 33 is a rear or posterior view of a sacrum according to another embodiment of the invention.

FIG. 34 is a reproduction of a fluoroscopic image of a sacroiliac view of a sacrum.

FIG. 35 is a flowchart illustrating a method according to an embodiment of the invention.

FIG. 36 is a flowchart illustrating another method according to an embodiment of the invention.

FIG. 37 is a flowchart illustrating yet another method according to an embodiment of the invention.

### DETAILED DESCRIPTION

Devices and methods for performing medical procedures within a sacral spine or “sacrum” are disclosed herein. In one variation, a method provides for the application of bone cement, such as polymethylmethacrylate (PMMA), along a load bearing area in a sacral ala region of a patient's spine. The PMMA can be used to mend a portion of a fractured area and/or reinforce a weakened load-bearing region. A method can include, for example, the use of a single-plane fluoroscopy device to navigate the access instrument to the sacral ala region of the patient. Anatomical landmarks can then be identified within the image to position an access tool within the sacral ala. Once the tool has been positioned, an expandable member, such as a balloon, can be inserted into the sacral ala region and expanded to define a void or cavity border within the sacral ala region. Such a cavity can define a low-pressure zone and a specific targeted region to be filled with PMMA. The low-pressure zone allows the PMMA to be filled in a concentrated and targeted area.

In one embodiment, a method includes percutaneously inserting a medical device via a medial-to-lateral approach at least partially into a sacral ala region of a spine based on a fluoroscopic image. An expandable portion of the medical device is then expanded to an expanded configuration while inserted within the sacral ala region such that a cavity is defined within the sacral ala region.

In another embodiment, a method includes imaging a spine with a fluoroscopy device to provide an anteroposterior view of the sacrum. A medical device is inserted in a medial-to-lateral direction into a sacral ala region of the sacrum to a location spaced laterally relative to a lateral-inferior border of a pedicle of the sacrum based on the image provided in the anteroposterior view. The spine is then imaged with the fluoroscopy device to provide a lateral view of the sacrum. A trajectory of the medical device is substantially aligned with a S1-S2 disc space of the sacrum based on the image provided in the lateral view of the sacrum.

In another embodiment, a method includes imaging a spine with a fluoroscopy device to provide a lateral-view image of the sacrum. At least three anatomical structures are identified within the lateral-view image. The at least three anatomical structures define a breach zone. A medical device is inserted at least partially into a sacral ala region of the sacrum such that the medical device does not enter the breach zone.

In another embodiment, a method includes imaging a spine of a patient with a fluoroscopy device to provide an anteroposterior view of the sacrum. An anatomical landmark is identified within the sacrum using the image. A medical device is inserted into the sacrum at an entry point associated with the anatomical landmark.

In another embodiment, a method includes identifying an anatomical landmark of a sacrum within an image having an anteroposterior view of a spine of the patient. An entry point into the sacrum is identified based on the anatomical landmark. At least three anatomical structures of the sacrum are identified within the image to define a breach zone. A medical device is inserted at least partially into a sacral ala region of the sacrum based on a location of at least one of the anatomical landmark or the breach zone.

In one embodiment, a kit includes a cannula configured to be inserted into a sacral ala region of a sacrum and an expandable device. The expandable device is configured to be inserted into the sacral ala region via a lumen of the cannula. The expandable device has a distal end portion that is configured to be expanded from a collapsed configuration to an expanded configuration within the sacral ala region such that a cavity is formed within the sacral ala region. In some embodiments, a kit can also include an access tool that is configured to provide an access path into a sacral ala region of a sacrum. In such an embodiment, the cannula can be configured to be inserted into the sacral ala region of the sacrum via the access path. In some embodiments, a kit can also include a delivery device that is movably disposable within the lumen of the cannula. The delivery device can be configured to inject bone cement into at least one of a lumen of the cannula or the cavity.

It is noted that, as used in this written description and the appended claims, the singular forms “a,” “an” and “the” include plural referents unless the context clearly dictates otherwise. Thus, for example, the term “a lumen” is intended to mean a single lumen or a combination of lumens. Furthermore, the words “proximal” and “distal” refer to direction closer to and away from, respectively, an operator (e.g., surgeon, physician, nurse, technician, etc.) who would insert the medical device into the patient, with the tip-end (i.e., distal end) of the device inserted inside a patient's body. Thus, for example, the catheter end inserted inside a patient's body would be the distal end of the catheter, while the catheter end outside a patient's body would be the proximal end of the catheter.

The term “tissue” is used here to mean an aggregation of similarly specialized cells that are united in the performance of a particular function. For example, a tissue can be a soft tissue area (e.g., a muscle), a hard tissue area (e.g., a bone structure), a vertebral body, an intervertebral disc, a tumor, etc.

The term “cannula” is used here to mean a component of an apparatus having one or more passageways configured to receive a device or other component. A cannula can be used to provide percutaneous access to an area within a patient's body. For example, a cannula can be substantially tubular. A cannula can be a variety of different shapes and size, such as having a round or octagonal outer perimeter.

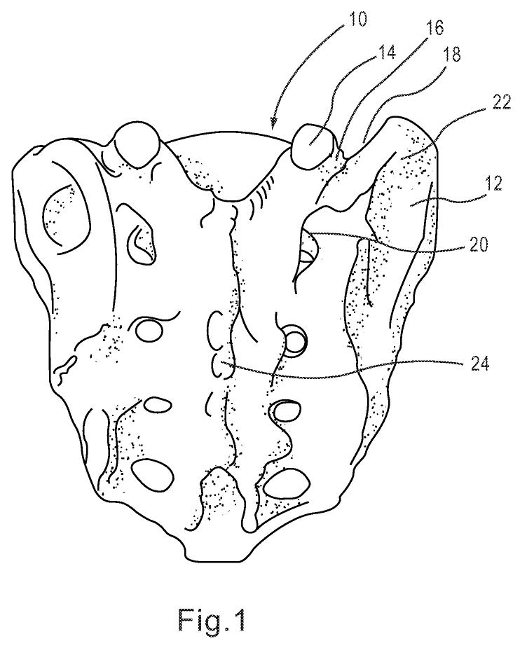

FIGS. 1-4 provide a brief summary of the anatomy of a sacral spine or sacrum. FIG. 1 is a rear or posterior view (as viewed from a back side of a patient), showing a dorsal surface of a sacrum 10. The sacrum 10 is a large irregular shaped bone that includes five vertebrae that have been fused together to form a single bone mass. The sacrum 10 has various portions including the sacroiliac joints 12, facet joints 14, and superior articular processes 16. The sacrum 10 is positioned between two iliac or pelvis bones and the interface between these bones forms the two sacroiliac joints 12. The sacrum 10 also defines V-shaped notches 18, and sacral foramen 20 (also referred to herein as “sacral foramina”), which are openings in the sacrum 10 that allow the pass-through of spinal nerve roots. The sacral ala 22 (also referred to herein as “sacral ala regions”) are the upper lateral portions of the sacrum 10, and a sacral crest 24 extends along the midline of the sacrum 10.

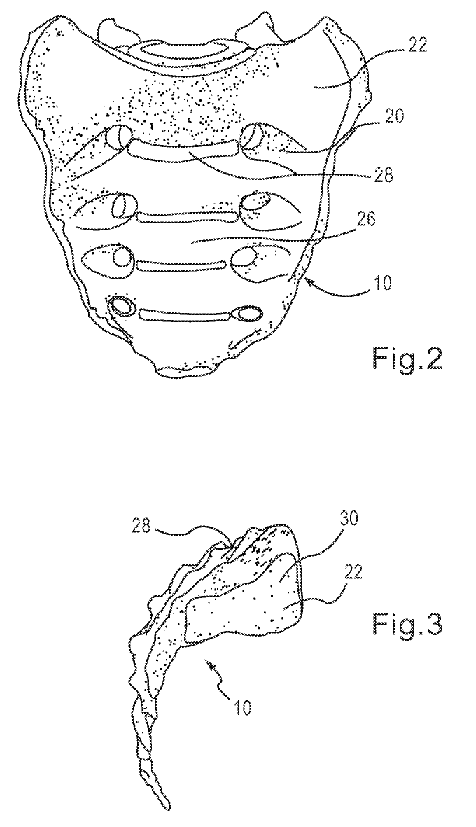

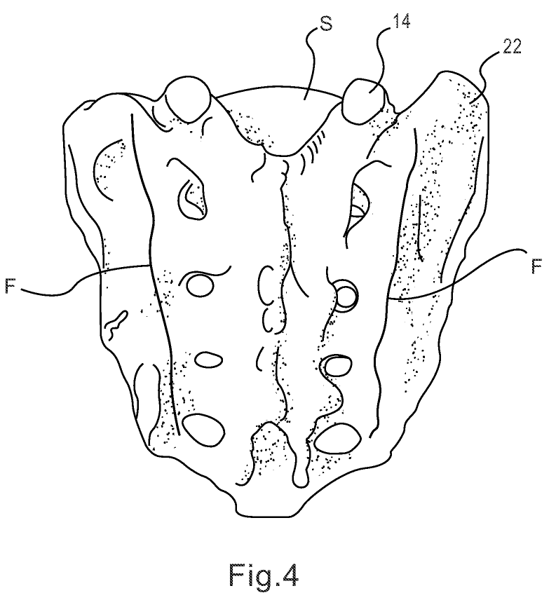

FIG. 2 is a front or anterior view (as viewed from a front side of a patient), showing a ventral surface of the sacrum 10. In this view, the sacral ala 22 are visible, as well as the sacral foramen 20. Also indicated in FIG. 2 is the sacral body 26 and a disc space referred to as the S1-S2 disc space 28. FIG. 3 is a side view illustrating a lateral view of the sacrum 10. In this view, a sacral anterior cortex 30 is visible, as well as the sacral ala 22 and the S1-S2 disc space 28.

FIG. 4 illustrates a sacrum S that has sacral fractures F running along both sides of the sacrum S. Typically, weight bearing areas within a sacrum include the sacral body (shown in FIG. 3), the facets 14, and sacral ala 22. The absence of condensation zones below the S2 disc indicates that weight transmission is typically achieved in an upper sacral region. Within the upper sacral region there is thin cortex and low bone density. The methods described herein can provide for fracture stabilization by targeting treatment to the largest weight bearing area of the sacral ala and by providing controlled bone cement delivery to these areas.

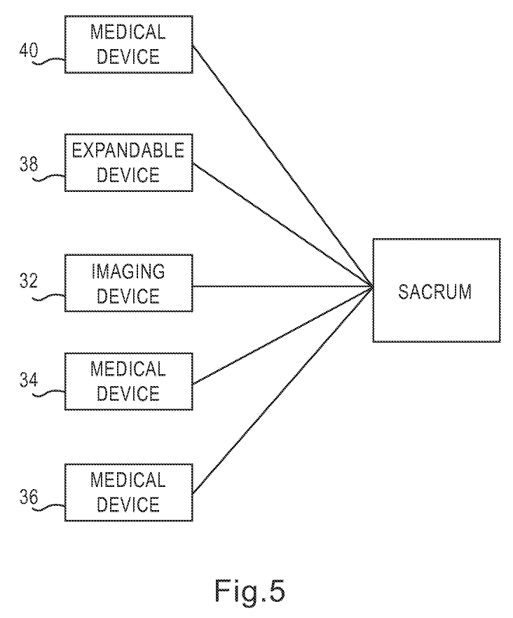

FIG. 5 is a schematic illustration of various medical devices that can used to perform the methods described herein. An imaging device 32, such as a single-plane fluoroscopy device, can be positioned to view an anteroposterior view (as illustrated in FIG. 1) of the sacrum of the patient's spine. While monitoring the images produced by the imaging device 32, a medical device 34 can be inserted into the sacral ala region of a the patient's spine S. The medical device 34 can be, for example, a guide wire, or a spine needle. Monitoring the images provided by the imaging device 32, in some embodiments, the medical device 34 can be positioned such that a trajectory of the device 34 is substantially in line with the S1-S2 disc space (not shown in FIG. 5) of the sacrum. In some embodiments, the medical device 34 can be used to mark or indicate an incision location on the patient. The specific advancement procedures are described in more detail below with reference to specific methods.

While continuing to monitor the images produced by the imaging device, another medical device 36, such as a stylet having a sharpened tip, can be inserted at the indicated location on the patient and positioned within the sacral ala region. For example, the medical device 36 can be disposed within a lumen of a cannula (not shown in FIG. 5), and the cannula and medical device 36 can be inserted into the sacral ala region. In some embodiments, an anatomical landmark is identified to guide the medical device 36 to the sacral ala region. In addition, a breach zone can be identified that is defined by anatomical structures or landmarks. The anatomical structures or landmarks indicate a region that should not be breached by the medical device 36. In some embodiments, the medical device 36 can be positioned such that a trajectory of the medical device 36 is substantially aligned with the S1-S2 disc space of the sacrum. In some embodiments, a device such as a drill can also be used to drill a channel, for example, to a border of the sacral body (not shown in FIG. 5). After the medical device 36 (or drill if used) has been removed from the cannula, an expandable device 38 having an expandable member (not shown in FIG. 5) can be inserted through the cannula and positioned within the sacral ala. The expandable member can be, for example a balloon, such as a balloon used in a Kyphoplasty procedure within a vertebral body. In alternative embodiments, the expandable device can be a mechanically expandable device, such as an expandable member formed for example, with biocompatible metals (e.g., titanium, nitinol, stainless steel, etc.).

The expandable device 38 can be inserted into the sacral ala region in a collapsed configuration and then moved to an expanded configuration while positioned within the sacral ala region. When the expandable device 38 is expanded, the expandable member will exert a pressure on an interior portion of the sacral ala region and form a void border or cavity border. After the cavity border has been formed, the expandable device 38 can be collapsed and removed from the sacral ala region, and a medical device 40, such as a bone cement delivery device, can be inserted into the sacral ala region. The medical device 40 can be used to inject or deliver a material, such as bone cement, into the cavity defined within the sacral ala region. For example, in some embodiments, PMMA bone cement is injected into the cavity. The bone cement inserted within the cavity can help stabilize a sacral fracture. Extravasation of bone cement into the sacral foramina can be a complication in such procedures. The methods described herein provide navigation of the various medical devices using the guidance of fluoroscopic imaging to provide controlled delivery of bone cement to targeted areas within the sacral ala region, and away from the sacral foramina.

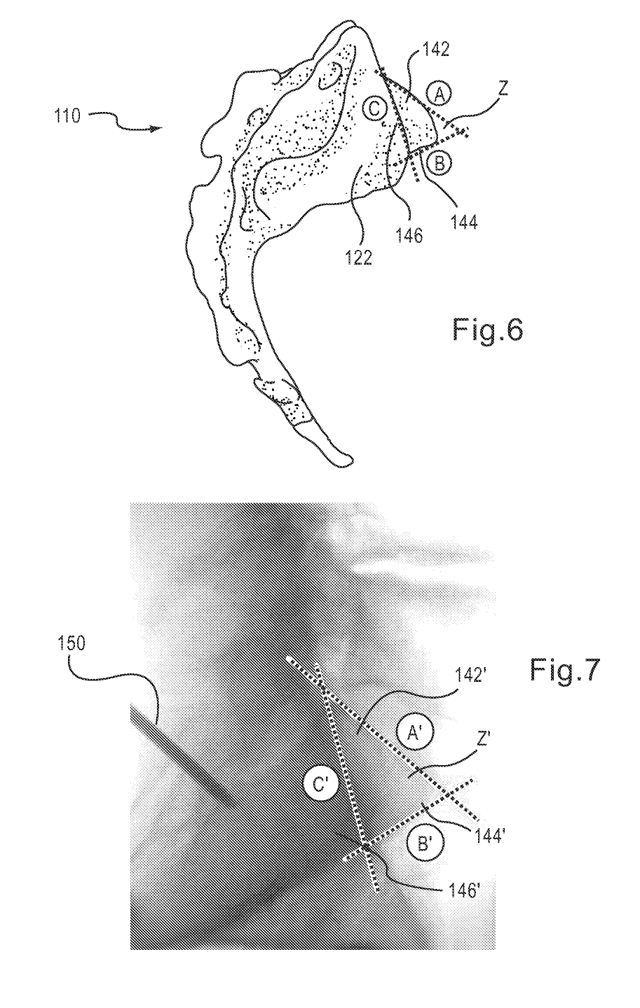

FIG. 6 is a side or lateral view of a sacrum 110 and illustrates a navigation method according to an embodiment of the invention. A method is provided for providing access to a sacral ala 122 through the guidance of a single fluoroscopic imaging device (not shown in FIG. 6), such as a C-arm computer tomography fluoroscopy (“C-arm”), which is a standard device used in many surgical facilities. The sacral ala 122 can be accessed by a medical device, such as an access tool, stylet, guidewire, surgical needle, cannula, etc. (not shown) through an approach that uses identifiable anatomical structures or “landmarks” to guide the medical device. As FIG. 6 illustrates, a “breach zone” Z can be identified within a lateral view of the sacrum 110. The breach zone Z is an area identified in an image that should not be compromised by a physician when placing instruments in the sacral ala 122. The breach zone Z is a projection defined by three intersecting borders, labeled A, B and C in FIG. 6, of three anatomical structures, as viewed in a lateral view of the sacrum. The three anatomical structures include a superior S1 endplate 142, an anterior wall of the sacral body 144, and an alar slope 146. FIG. 7 illustrates a projection as viewed in an actual fluoroscopic image. The projection includes borders A′, B′, and C′ defined by a superior S1 endplate 142′, an anterior wall of the sacral body 144′, and an alar slope 146′. The use of anatomical landmarks to assist in the placement of instruments, such as medical instrument 150 shown in FIG. 7, within the sacral ala is further described below with reference to further embodiments of the invention.

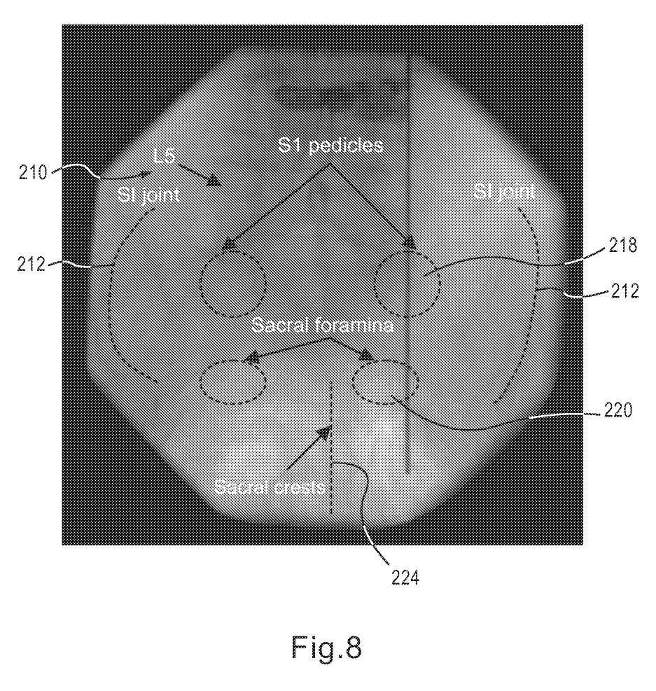

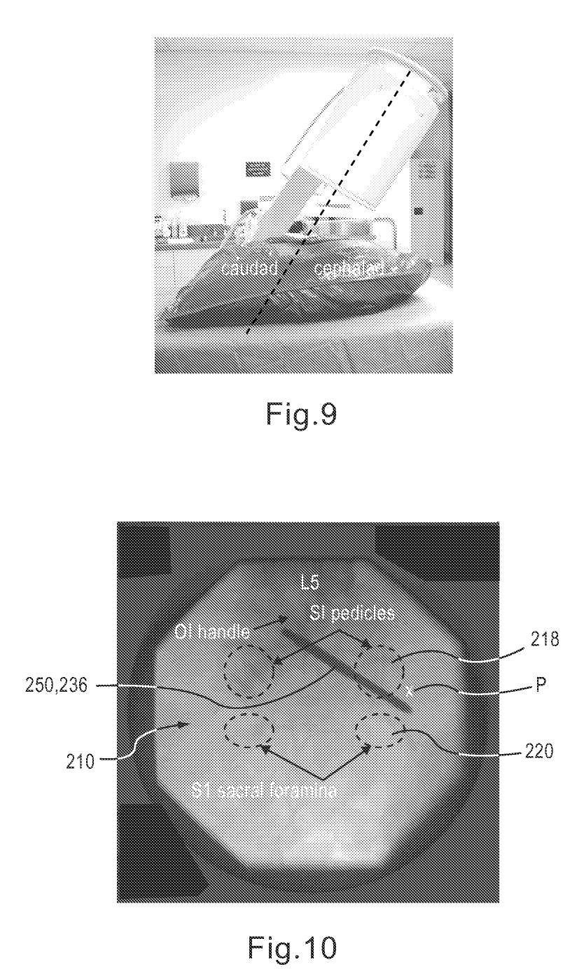

FIGS. 8-20 illustrate the various steps associated with a method according to an embodiment of the invention. In this embodiment, a method is provided that uses the guidance of fluoroscopic imaging to identify a pedicle of a sacrum to use as a landmark to guide medical tools within the sacrum. The method can be used to treat a sacral fracture (e.g., as shown in FIG. 4) and includes first positioning a patient in a prone position. Pads or other lifts can be placed under the patient's pelvis to caudalize the sacral orientation. A fluoroscopy device is positioned to focus on the patient's sacrum 210 and to provide an anteroposterior (AP) view of the sacrum 210. In this position, the sacral crest 224 can be identified within the sacrum 210 within the image. The S1 pedicles 218, S1 joint 212 and the sacral foramina 220 are also made visible, as shown in FIG. 8. A C-arm can be positioned cephalically to compensate for the angle of the S1 superior endplate. FIG. 9 illustrates an example of the C-arm positioning.

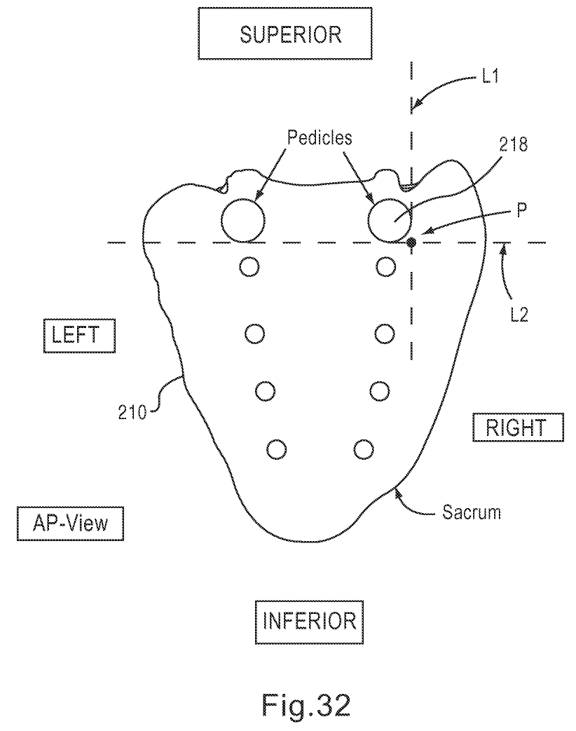

An entry point into the bone can be made just lateral to the lateral inferior border of the pedicle, as shown at P in FIG. 10. For example, as shown in FIG. 32, a first line L1 can be drawn on the patient's skin that corresponds to a lateral border of the pedicle 218, and a second line L2 can be drawn on the patient's skin that corresponds to an inferior border of the pedicle 218. The desired entry point P into the bone will be at the intersection of Line L1 and line L2. In some embodiments, a skin incision can be made, for example, 1 cm medial and 1 cm superior to the bone entry point because the access instruments are inserted in a medial-to-lateral direction towards the fractured ala. Thus, in this embodiment, the pedicle 218 is used as a landmark reference for the entry point P. The bone (e.g., the sacrum) can then be accessed using a medical device 236, such as an osteointroducer (e.g., a stylet) having a diamond- or bevel-tip configuration. For example, the medical device 236 can be inserted through a lumen of a cannula 250, as shown in FIG. 10. In some embodiments, a lateral angle of the device 236 can be approximately 40-45° from a midline of the sacrum 210.

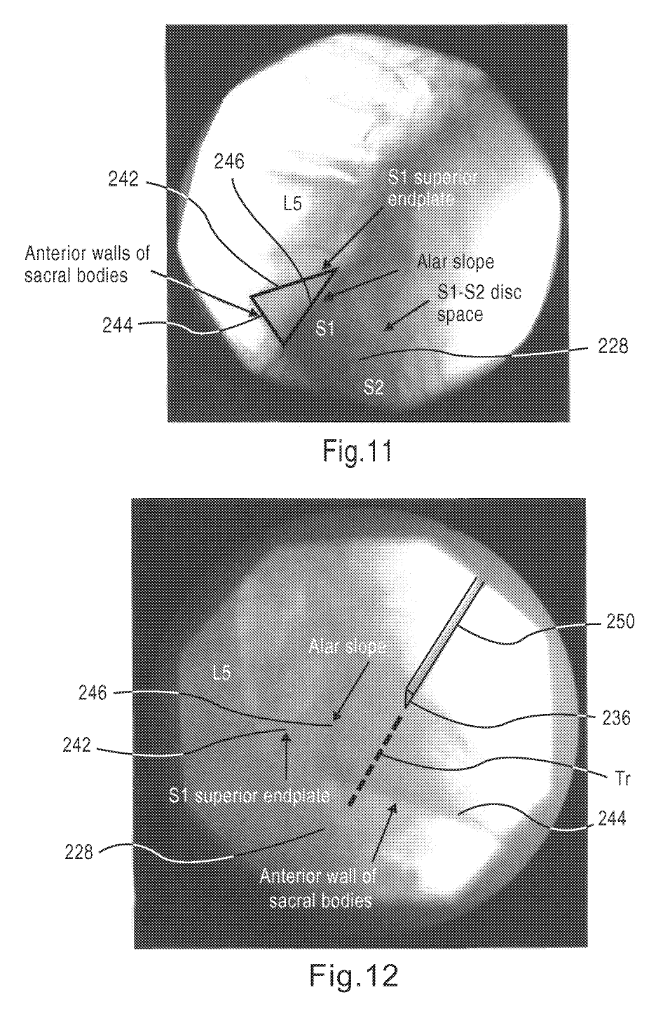

The fluoroscope is then adjusted to produce a lateral view of the sacrum 210. As shown in FIG. 11, in the lateral view, the edges of the S1 superior endplate 242 can appear to be superimposed in the image so that no “halos” are present and the anterior walls of the sacral bodies 244 can be viewed. The alar slope 246, which is the radiographic projection in the lateral view of the antero-superior ala as it intersects within the auricular surface can also appear to be superimposed within the image. The S1-S2 disc space 228 can also be present within the image. As shown in FIG. 12, a lateral angle of a trajectory Tr of the device 236 can be aligned approximately with the S1-S2 disc space 228.

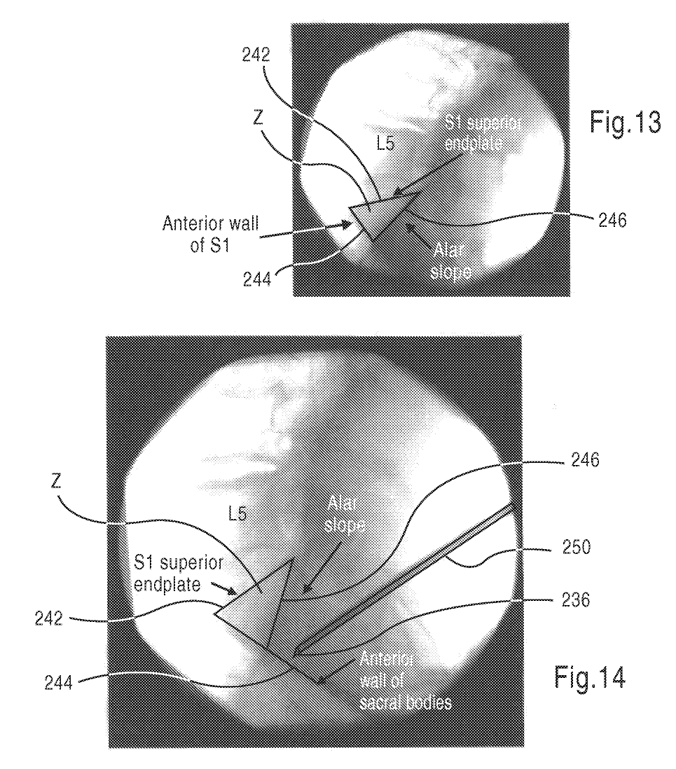

As described above, a breach zone Z can be identified in the lateral view that is defined by anatomical structures or landmarks, such as the S1 superior endplate 242, the anterior wall of the sacral bodies 244, and the alar slope 246, as shown in FIG. 13. In the lateral view, the breach zone Z identifies an area within the sacrum in which no device (e.g., the medical device 236) should be inserted during a procedure. The alignment of the medical device 236 with the S1-S2 disc space 228 in the lateral view ensures that the breach zone Z is not entered in this medial to lateral approach. If the S1-S2 disc space 228 is not visible, the trajectory Tr (FIG. 12) should be inferior to the breach zone Z. Once the trajectory Tr is defined, the medical device 236 can be advanced to, for example, approximately 3-4 mm posterior to the anterior wall of the sacral bodies 244, as shown in the lateral view of FIG. 14. The medical device 236 can then be removed from the cannula 250.

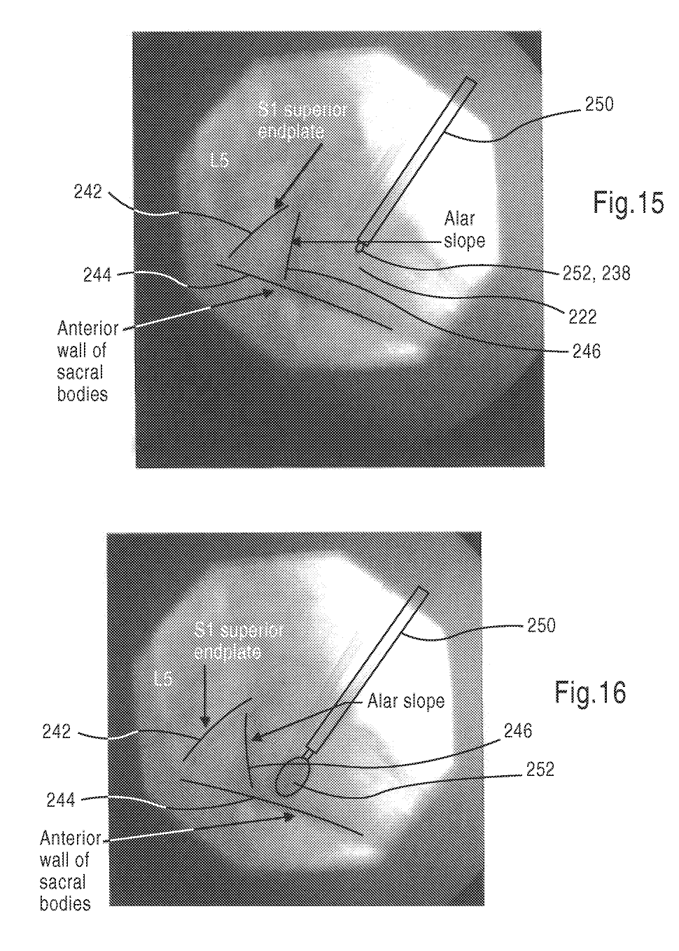

Next, an expandable device 238 is inserted through a lumen of the cannula 250, as shown in FIGS. 15 and 16. In this embodiment, the expandable device 238 includes an expandable member 252 in the form of a balloon at a distal end of the expandable device 238. The cannula 250 can be withdrawn (moved proximally) as needed until a distal end of the expandable device 238 (e.g., the expandable member 252) is visible within the sacral al region 222, as shown in FIG. 15. For example, the expandable member 252 can have a radiographic marker that can be viewed with the fluoroscopic imaging device. The expandable member 252 can be inflated while inserted within the sacral ala region, as shown in FIG. 16. In some embodiments, the expandable member 252 is expanded in 0.5 cubic centimeter (cc) increments under continuous fluoroscopic monitoring in the lateral view.

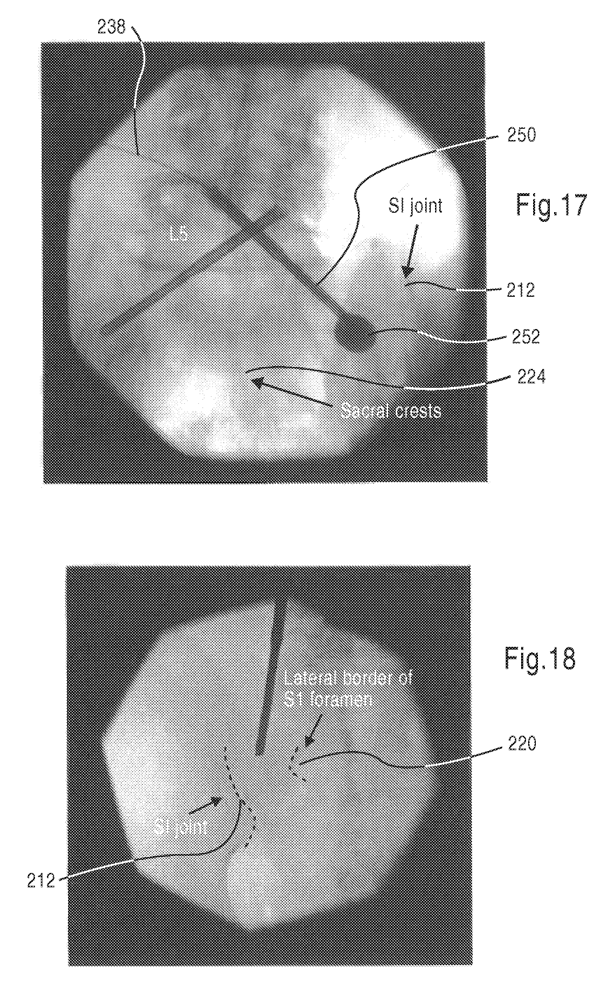

An AP view as described above and/or a sacroiliac (SI) view can be used to check the proximity of the expandable member 252 to the sacral foramina 220 and the sacroiliac joint 212. FIG. 17 is an AP view showing the proximity of the expandable member 252 in relation to the sacroiliac joint 212. A second cannula 250′ is visible in the image of FIG. 17. For example, a second cannula 250′ can be inserted on a contra-lateral side of the sacrum. FIG. 18 is a SI view showing the proximity of the expandable member 252 to the lateral border of the foramen 220 and the sacroiliac joint 212. A SI view is an oblique view used to visualize the sacroiliac joint 212. A C-arm image intensifier looks directly down the sacroiliac joint 212, with the C-arm rotated approximately 15°-20° contralaterally, away from the side being accessed.

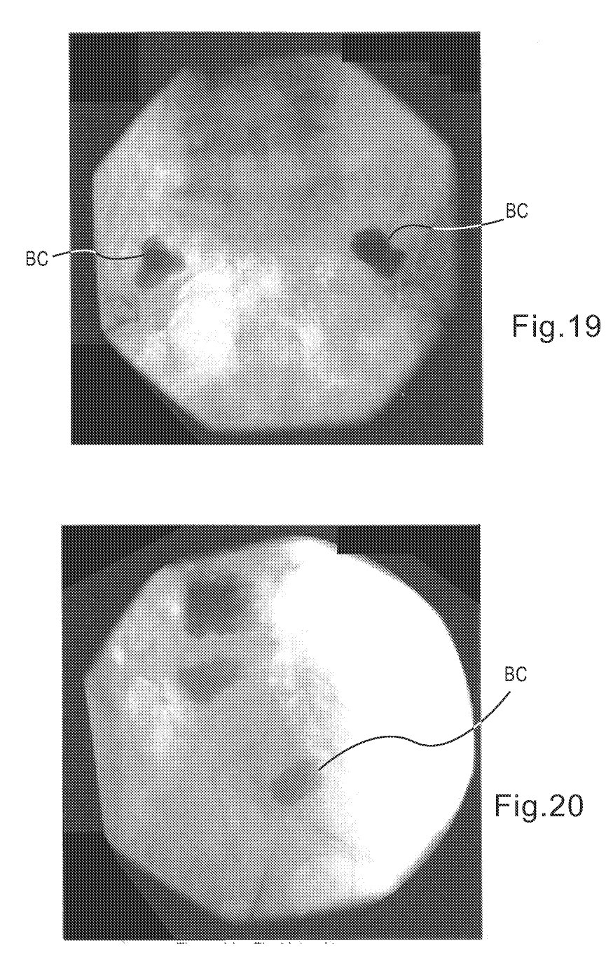

The expansion of the expandable member 252 (e.g., as shown in FIGS. 16 and 17) compresses the cancellous bone within the sacral ala region 222 and defines a cavity border or void border within an interior of the sacral ala region 222. After the cavity border has been defined, the expandable device 238 is collapsed and removed from the sacrum 210 and a medical device (not shown), such as a bone cement delivery device, is inserted through the cannula 250 and into the sacral ala 222. The medical device is used to inject bone cement BC (shown in FIGS. 19 and 20), such as PMMA, into the cavity. The bone cement BC is injected in a viscous, doughy state (e.g., dull and toothpaste-like in appearance). The bone cement BC can be injected incrementally, such as in 0.5 cc increments. During injection, lateral (e.g., FIG. 20) and SI views can be monitored to watch for signs of cement extravasion anteriorly, superiorly, or medially into the sacral foramina. The injection process should be discontinued if cement extravasion into the sacral foramina, or outside the anterior or superior ala is observed. In some embodiments, the volume of bone cement should not exceed the volume of the expanded expandable member (e.g., the balloon), or in other words, should not exceed the volume of the cavity created within the sacral ala.

Once the bone cement BC has been disposed within the cavity, the medical device (e.g., bone cement delivery device) is removed from the sacrum 210 and another device, such as an empty bone cement delivery device, is used to tamp down any potential cement bolus tail extending into the posterior aspect of the sacrum in the lateral view. For example, a plunger within an empty bone cement delivery device can be used to push down a bolus tail. Other devices can alternatively be used to tamp down any cement bolus. The cannula 250 is then removed from the sacrum 210. Final AP and lateral view images can be taken, which show the bone cement BC disposed within the sacral ala, as shown in FIGS. 19 and 20, respectively. Although the procedures above have been described as being performed on one side of the sacrum, it should be understood that the opposite side of the sacrum (e.g., the contralateral sacral ala) can also be treated in the same manner simultaneously, or sequentially.

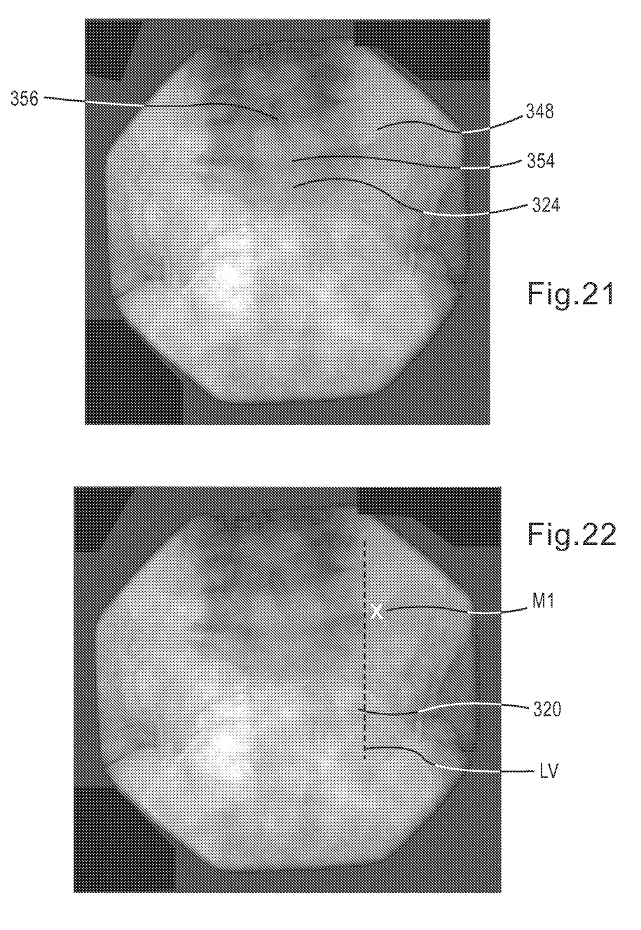

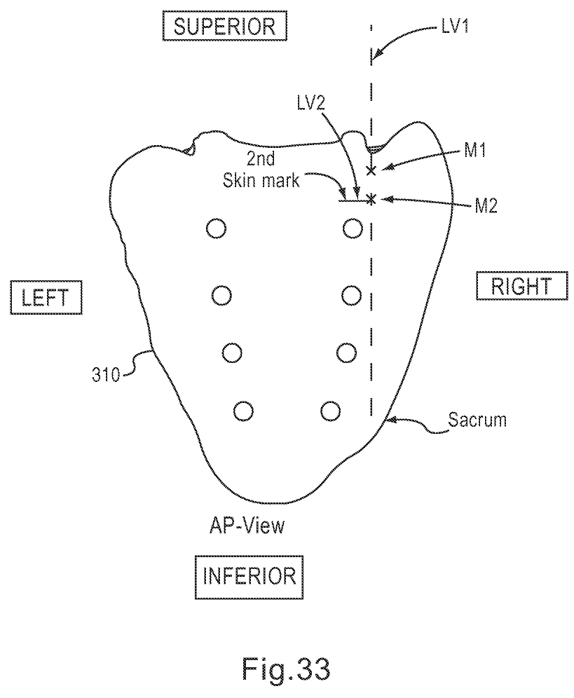

FIGS. 21-31 illustrate a method of performing a sacroplasty procedure according to another embodiment of the invention. In this embodiment, the V notch defined by the sacrum is used as an anatomical reference to assist in guiding the insertion of medical instruments. As with the previous embodiment, a patient is first placed in a prone position. In an AP view using fluoroscopic imaging, the L5 inferior endplate 354, L5 spinous process 356, V notch 348 (defined by the lateral wall of the S1 superior articular process and medial aspect of the sacral ala), and the sacral crestline 324 are identified as shown in FIG. 21. The V notch 348 is located lateral to the L5 inferior endplate 354. FIG. 22 includes a dashed-line LV indicating a lateral position of the V notch 348 in the AP view. After identifying the V notch 348, a first mark M1 can be placed on the skin of the patient at a location, for example, approximately 5 mm from the V notch to ensure needle alignment lateral to the S1 foramen 320, as shown in FIGS. 22 and 33. In some cases, the fluoroscope may need to be adjusted to minimize “halo” on these identified anatomical landmarks.

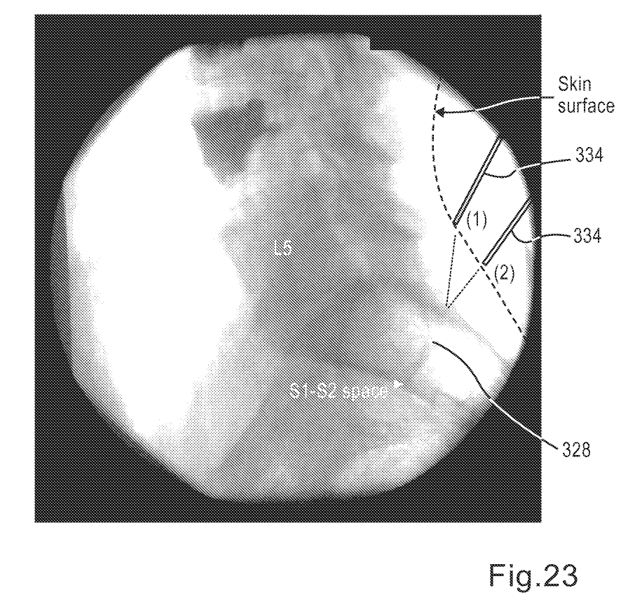

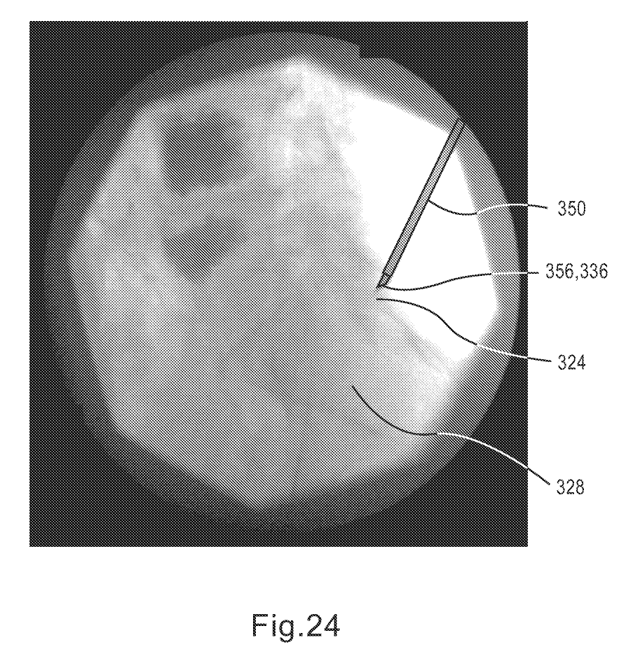

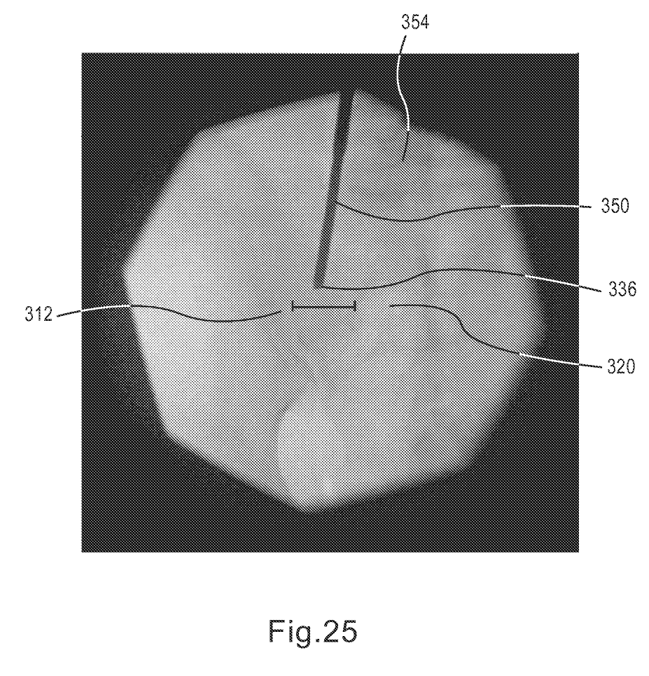

The C-arm of the fluoroscope can then be adjusted to obtain a lateral view as shown in FIG. 23. A medical device 334, such as a spine needle or guidewire, can then be placed on the skin mark M1 corresponding to the location of the V notch at a first position labeled 1 in FIG. 23. A vertical line LV1 is drawn through the mark M1 on the skin, as illustrated in FIG. 33. The placement of the medical device 334 can then be adjusted to a second position, labeled position 2 in FIG. 23, such that the medical device 334 is substantially in line with the S1-S2 disc space 328. A second mark M2 is placed on the patient's skin corresponding to the S1-S2 disc space 328. Mark M2 is identified at the intersection or junction of the adjusted position of the medical device 334 (indicated at LV2 in FIG. 33) and the line LV1 associated with the V notch mark M1, as shown in FIG. 33. An incision can then be made at mark M2 (i.e., the intersection of line LV1 and LV2). A medical device, such as a stylet or osteointroducer, can then be inserted therein. As shown in FIG. 24, a medical device 336 can be inserted through a lumen of a cannula 350. A tip 356 of the medical device 336 is advanced and oriented such that it is substantially in line with the S1-S2 disc space 328, as shown in FIG. 24. The tip 356 can then be docked along the lateral sacral crest 324. A handle of the device 336 can be rotated medially 15-20° (from sacral midline) such that a trajectory of the device 336 is oriented medial-to-lateral. The medical device 336 is then advanced so that it is securely positioned in the sacral ala, for example, advanced approximately 5 mm. The C-arm of the fluoroscope is then adjusted to a Ferguson view relative to the L5 inferior endplate (e.g. 35 degrees cranio-caudal angle) then adjusted to an oblique view approximately at a 15 degree angle, to position the scope for a cranio-caudal, oblique view, otherwise referred to as a SI view, as shown in FIG. 25. It is noted that the C-arm angle is defined based on the sacral anatomy position, not the operating table. The position of the tip 356 of the medical device 336 can be verified to ensure that it is approximately halfway from the foramen 320 lateral border and the sacroiliac joint 312 medial border. The tip 356 can be adjusted as necessary.

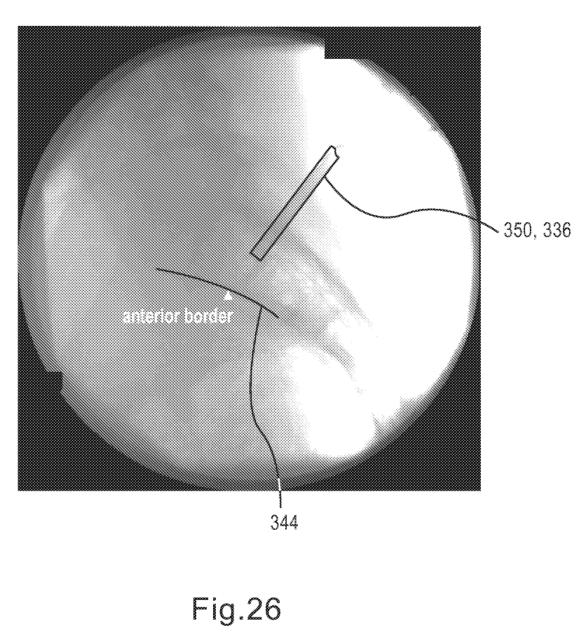

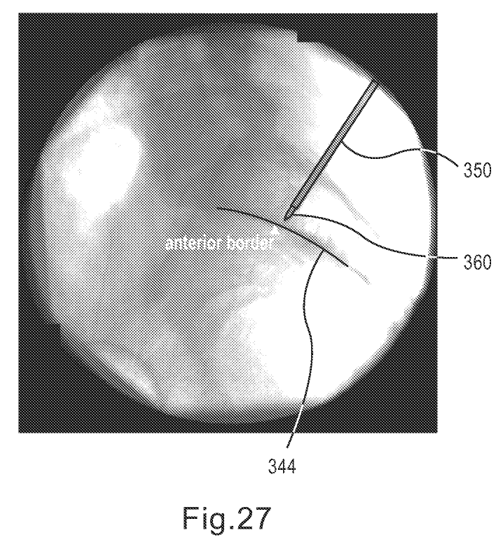

Next, the scope is moved to obtain a lateral view. The location of the medical device 336 (e.g., a stylet) along the S1-S2 disc space 328 is then verified. If needed, the medical device 336 can be adjusted to ensure proper trajectory alignment along the S1-S2 disc space 328. The medical device 336 is then advanced midway to the sacral body anterior border 344, as shown in FIG. 26. In this embodiment, the medical device 336 is removed and an optional twist drill 360 is inserted through the cannula 350, as shown in FIG. 27. The drill 360 is used to drill a channel that is, for example, 2-3 mm posterior to the anterior border of the sacral body 344. The twist drill 360 is then removed from the sacrum.

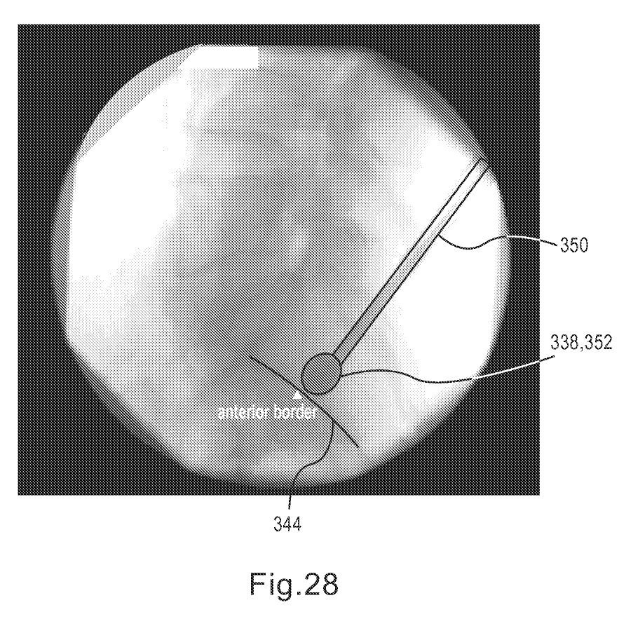

With the cannula 350 properly positioned within the sacrum, an expandable device 338 is inserted through the cannula 350 in a collapsed configuration (not shown) and into the sacral ala, as shown in FIG. 28. The expandable device 338 includes an expandable balloon 352 at a distal end (shown expanded in FIG. 28). The expandable device 338 is advanced until radiographic bands on the balloon 352 have exited a distal end of the cannula 350 and are visible on the fluoroscopic image. For example, the balloon 352 can be disposed outside of the cannula 350 approximately 2 mm posterior to the anterior border 344. The balloon 352 can then be inflated, for example, to 1 cc to anchor the balloon 352 to the trabecular surface. In some embodiments, a stylet coupled to the balloon 352 can then be removed. The balloon 352 can then be further inflated to, for example, 3 ccs, or to a point when the balloon 352 appears to contact the anterior border 344 as viewed within the fluoroscopic image. The expansion of the balloon 352 compresses cancellous bone within the sacral ala and defines a void border or cavity border.

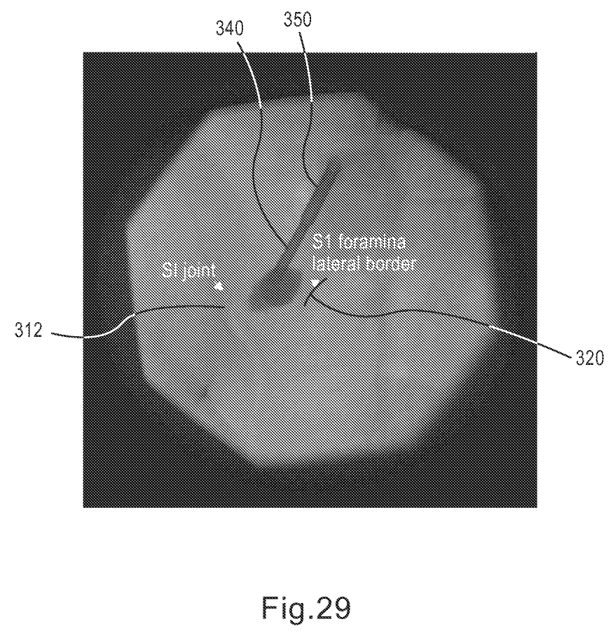

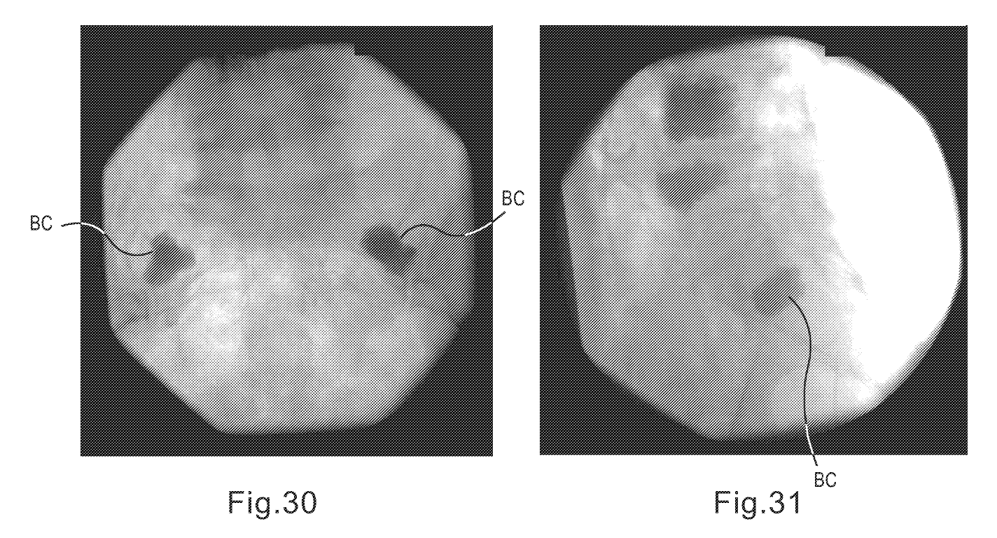

After expanding the balloon 352, the balloon 352 is deflated or collapsed and removed from the sacral ala. A bone filler device 340 is then inserted through the cannula 350 and into the sacral ala as shown in FIG. 29. The fluoroscopy device is then switched to a SI view and the bone filler device 340 injects or delivers bone cement BC to the cavity defined within the interior of the sacral ala, as shown in FIG. 29. Using the SI image, the delivery of bone cement BC is monitored to ensure that the bone cement BC does not encroach on to the S1 foramina 320. As with the previous embodiment, a final check can be performed by viewing both an AP view (FIG. 30) and a lateral view (FIG. 31) of the sacrum, which illustrate bone cement BC within the sacral ala.

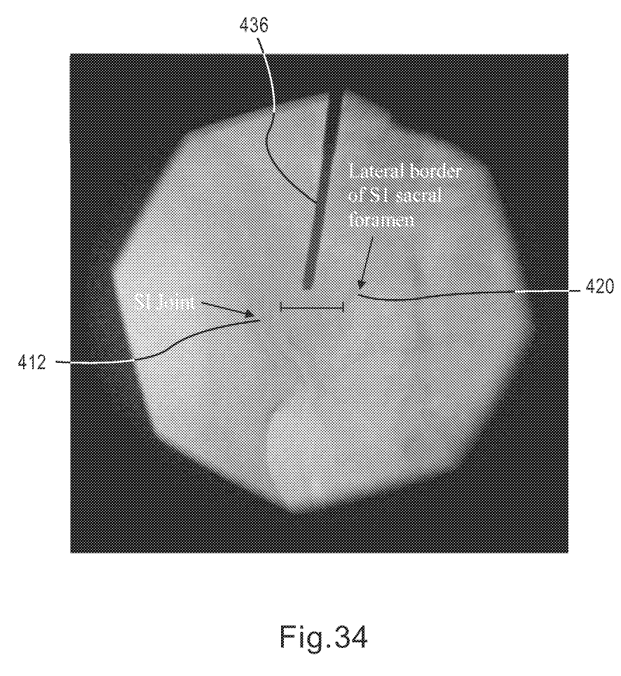

FIG. 34 illustrates another method of guiding a medical device within a sacrum. In this embodiment, two anatomical landmarks are used to assist in identifying an entry point into a sacrum and guiding the insertion of medical instruments into the sacrum. As with the previous embodiment, a patient is first placed in a prone position. FIG. 34 is a fluoroscopic image of sacroiliac view of a sacrum. In this example, the two anatomical landmarks are the S1 foramen 420 of the side being accessed and the sacroiliac (SI) joint 412. The anatomical landmarks can be visualized utilizing an oblique or sacroiliac view of the sacrum (as shown in FIG. 34) with the C-arm angled to look along an angle of the SI joint 412. The C-arm can be angled, for example, at 15-30 degrees away from the side being accessed.

In this position, the SI joint 412 and a lateral border of the corresponding sacral foramen 420 are visible. A skin incision can be made at the level of the sacral foramen at a point that is midway between the SI joint 412 and the lateral border of the of the sacral foramen 420. As with the previous embodiments, while using fluoroscopic imaging, an access instrument 436 can be inserted through the skin incision and docked at on the posterior aspect of the bone. Once the instrument is docked on the bone, the C-arm can be switched to a lateral view. In the lateral view, the same procedures described above for previous embodiments can then be performed. For example, an instrument trajectory can be aligned with a S1-S2 disc space, and a breach zone can be identified as described for previous embodiments. Also as described above, an expandable device (not shown) can be inserted into the sacral ala region and expanded to create a cavity. Bone cement can then be injected into the cavity.

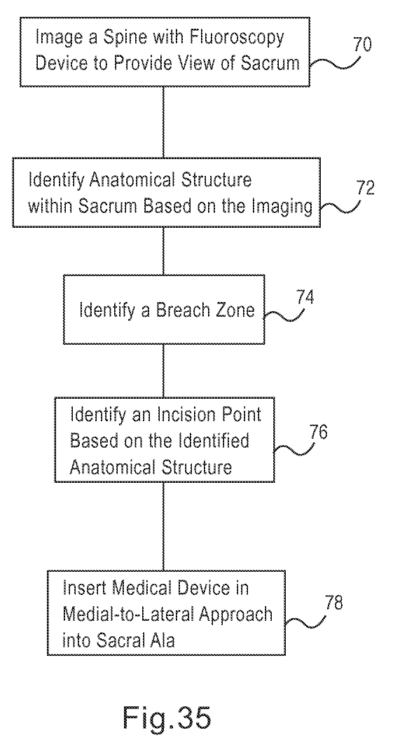

FIG. 35 is a flowchart illustrating a method for performing a medical procedure within a spine according to an embodiment of the invention. The method includes at 70, imaging a spine with a fluoroscopy device to provide a view of the sacrum. An anatomical landmark can be identified based on the imaging at 72. The anatomical landmark can be, for example, a pedicle of the sacrum. At 74, a breach zone can be identified. The breach zone can be defined, for example, by a superior S1 endplate, an anterior wall of a sacral body, and an alar slope in a lateral view of the sacrum. The anatomical landmark is used to identify an incision or entry point for a medical device at 76. At 78, a medical device is inserted at the incision point in a medial-to-lateral approach into a sacral ala region of the sacrum to perform a medical procedure within the sacral ala.

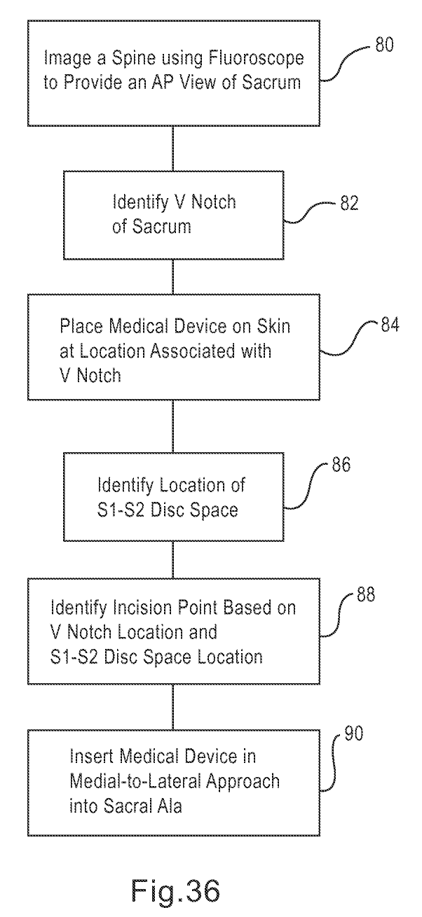

FIG. 36 is a flowchart illustrating a method for performing a medical procedure within a spine according to another embodiment of the invention. The method includes at 80, imaging a spine using a fluoroscopy device to provide an AP view of the sacrum. At 82, a V notch of the sacrum is identified. A medical device is placed on the patient's skin at a location corresponding to a location associated with the V notch at 84. For example, a vertical line can be drawn on the patient's skin at a location approximately 5 mm lateral from the V notch to ensure needle alignment lateral to a S1 foramen of the sacrum. At 86, an S1-S2 disc space of the sacrum is identified and the medical device is adjusted to a location on the skin such that it is substantially in-line with the S1-S2 disc space. At 88, an incision or entry point is identified based on the location associated with the V notch and the location associated with the S1-S2 disc space. For example, an incision point can be identified at an intersection of the vertical line associated with the V notch and the adjusted location of the medical device. At 90, a medical device can be inserted in a medial-to-lateral approach at the identified incision point to perform a medical procedure within the sacral ala.

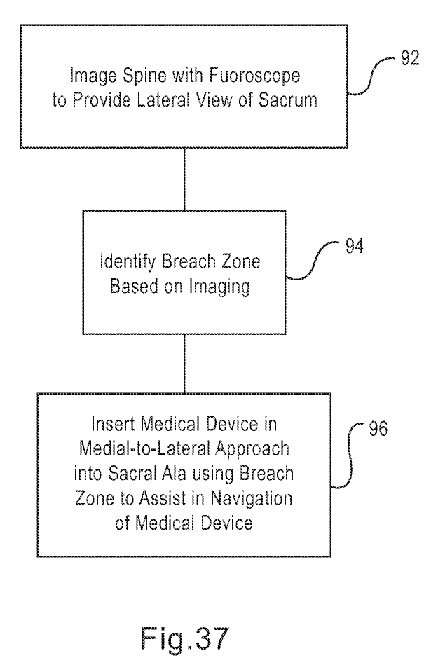

FIG. 37 illustrates another method according to an embodiment of the invention. The method includes at 92, imaging a sacrum of a spine using fluoroscopy to provide a lateral image of the sacrum. A breach zone defined by a superior endplate, an anterior wall of the sacral body, and an alar slope, is identified based on the imaging, at 94. At 96, a medical device can be inserted in a medial-to-lateral approach at least partially into a sacral ala region of a spine using the identified breach zone to assist in navigation of the medical device.

As described above, various medical devices can be used in combination to perform the medical procedures described herein. Other medical devices not specifically described can be used in addition to, or alternatively to, the example medical devices described herein. Further, the medical devices for any of the embodiments may be constructed with any suitable material used for such medical devices. For example, the medical devices can each be formed with a biocompatible material, such as stainless steel, titanium or suitable plastic materials, such as various polymers. The expandable member (e.g., balloon) can be formed with various flexible or expandable materials such as plastics (e.g., various polymers) and/or rubber materials having flexible or expandable characteristics.

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

For example, although the above methods have been described with reference to inserting an expandable member into the sacral ala to subsequently inject bone cement into the sacral al, the above methods can be used to guide other types of medical devices to a location within a sacrum and/or to perform other procedures. For example, in some embodiments, the methods can be used to guide only a bone cement filling device to a location within the sacrum to inject bone cement into the sacral ala (e.g., without having previously performed a procedure with an expandable device). In another example, other types of expandable devices can be used in place of a balloon-type expandable member. In addition, other types of access devices can be used, for example, instead of a stylet disposed within a cannula. For example, a cannula having a sharpened distal end can be inserted into the sacral ala. Such a cannula can include a blocking device, such as a plunger within a lumen of the cannula, to block tissue from entering the lumen of the cannula while being inserted into the sacrum.

What is claimed is:

1. A method comprising:
imaging a spine with a fluoroscopy device to provide a lateral-view image of the sacrum;
identifying at least three anatomical structures within the lateral-view image, the at least three anatomical structures defining a breach zone; and
inserting a medical device at least partially into a sacral ala region of the sacrum such that the medical device does not enter the breach zone.

2. The method of claim 1, wherein the medical device is an access tool, the method further comprising:
after the inserting, aligning the access tool with a S1-S2 disc space.

3. The method of claim 1, wherein the medical device includes an expandable portion, the method further comprising:
after the inserting, expanding the expandable portion of the medical device to an expanded configuration such that a cavity is formed within the sacral ala region.

4. The method of claim 1, wherein the medical device includes an expandable portion, the method further comprising:
after the inserting, expanding the expandable portion of the medical device to an expanded configuration such that a cavity is formed within the sacral ala region; and
injecting a bone cement into the cavity.

5. The method of claim 1, wherein the breach zone is defined at least in part by a superior endplate, an anterior wall of a sacral body, and an alar slope of the sacrum.

6. The method of claim 1, wherein the inserting includes percutaneously inserting the medical device.

7. The method of claim 1, wherein the medical device includes an expandable portion, the method further comprising:
after the inserting, expanding the expandable portion of the medical device to an expanded configuration such that a cavity is formed within the sacral ala region and a low-pressure zone within the sacral ala region is formed.

8. A method, comprising:
imaging a spine of a patient with a fluoroscopy device to provide an anteroposterior view of the sacrum;
identifying an anatomical landmark within the sacrum using the image; and
inserting a medical device into the sacrum at an entry point associated with the anatomical landmark.

9. The method of claim 8, wherein the anatomical landmark is a V notch of the sacrum.

10. The method of claim 8, wherein the anatomical landmark is a pedicle of the sacrum.

11. The method of claim 8, wherein the anatomical landmark is a first anatomical landmark, the method further comprising:
identifying a second anatomical landmark within the sacrum, the entry point being associated with the first anatomical landmark and the second anatomical landmark.

12. The method of claim 8, wherein the anatomical landmark is a first anatomical landmark, the method further comprising:
identifying a second anatomical landmark within the sacrum, the first anatomical landmark is a sacral foramen, the second anatomical landmark is a sacroiliac joint.

13. The method of claim 8, wherein the identifying includes:
drawing a first line on a portion of skin, the first line corresponding to a lateral border of a pedicle of the sacrum;
drawing a second line on a second portion of skin, the second line corresponding to an inferior border of the pedicle; and
identifying an intersection point between the first line and the second line, the intersection point corresponding to the entry point for inserting a medical device into the sacrum.

14. A method of claim 8, wherein the anatomical landmark is a first anatomical landmark, the method further comprising:
identifying a second anatomical landmark within the sacrum, the first anatomical landmark is a sacral foramen, the second anatomical landmark is a sacroiliac joint; and
making an incision at a level of the identified sacral foramen at a point midway between the identified sacroiliac joint and the identified sacral foramen.

15. A method, comprising:
percutaneously inserting a medical device via a medial-to-lateral approach at least partially into a sacral ala region of a spine based on a fluoroscopic image; and
expanding an expandable portion of the medical device to an expanded configuration while inserted within the sacral ala region such that a cavity is formed within the sacral ala region.

16. The method of claim 15, further comprising:
after the expanding, injecting a bone cement into the cavity.

17. The method of claim 15, further comprising:
identifying at least three anatomical structures within the fluoroscopic image, the at least three anatomical structures defining a breach zone within the sacra ala region.

18. The method of claim 15, further comprising:
identifying in a lateral view of a fluoroscopic image a breach zone in the sacra ala region, the breach zone defined at least in part by a superior endplate, an anterior wall of the sacral body, and an alar slope.

19. The method of claim 15, wherein the expanding includes forming a low-pressure zone within the sacral ala region.

20. The method of claim 15, further comprising:
prior to the inserting, aligning a trajectory of an access device with the S1-S2 disc space of the spine.

21. The method of claim 15, further comprising:
after the expanding, injecting a bone cement into the cavity to treat a fracture within the sacrum.

22. The method of claim 15, further comprising:
prior to the percutaneously inserting, imaging the sacrum to provide a sacroiliac view or an anteroposterior view; and
identifying a sacral foramina and the sacroiliac joint based on the sacroiliac view or the anteroposterior view.

23. A method, comprising:
imaging a spine with a fluoroscopy device to provide an anteroposterior view of the sacrum;
inserting a medical device in a medial-to-lateral-direction into a sacral ala region of the sacrum to a location spaced laterally relative to a lateral inferior border of a pedicle of the sacrum based on the image provided in the anteroposterior view;
after the inserting, imaging the spine with the fluoroscopy device to provide a lateral view of the sacrum; and
substantially aligning a trajectory of the medical device with a S1-S2 disc space of the sacrum based on the image provided in the lateral view of the sacrum.

24. The method of claim 23, further comprising:
identifying a breach zone in the lateral view, the breach zone defined at least in part by a superior endplate, an anterior wall of a sacral body, and an alar slope of the sacrum.

25. The method of claim 23, further comprising:
after the substantially aligning, inserting an expandable device into the sacral ala region of the sacrum.

26. The method of claim 23, further comprising:
after the substantially aligning, inserting an expandable device into the sacral ala region of the sacrum; and
expanding the expandable device.

27. The method of claim 23, further comprising:
after the substantially aligning, inserting an expandable device into the sacral ala region of the sacrum; and
expanding the expandable device until the expandable device contacts at least one of a cortical wall, an anterior ala, a superior ala, a sacral foramen or a sacroiliac joint of the sacrum.

28. The method of claim 23, further comprising:
after the substantially aligning, inserting an expandable device into the sacral ala region of the sacrum; and
expanding the expandable device until the expandable device has an interior volume of approximately 3 cubic centimeters.

29. The method of claim 23, further comprising:
after the substantially aligning, inserting an expandable device into the sacral ala region of the sacrum; and
expanding the expandable device until the expandable device has an interior pressure of approximately 400 psi.

30. The method of claim 23, further comprising:
after the substantially aligning, inserting an expandable device into the sacral ala region of the sacrum; and
expanding the expandable member in 0.5 cubic centimeter increments under continuous fluoroscopy monitoring.

31. The method of claim 23, further comprising:
imaging the sacrum to provide a sacroiliac view or an anteroposterior view; and
identifying a sacral foramina and the sacroiliac joint based on the sacroiliac view or the anteroposterior view.

32. A method, comprising:
identifying an anatomical landmark of a sacrum within an image having an anteroposterior view of a spine of the patient;
identifying an entry point into the sacrum based on the anatomical landmark;
identifying at least three anatomical structures of the sacrum within the image to define a breach zone; and
inserting a medical device at least partially into a sacral ala region of the sacrum based on a location of at least one of the anatomical landmark or the breach zone.

33. The method of claim 32, further comprising:
expanding at least a portion of the medical device while disposed within the sacral ala region of the sacrum.

34. The method of claim 32, further comprising:
expanding at least a portion of the medical device such that a cavity is formed within the sacral ala region of the sacrum; and
injecting bone cement into the cavity within the sacral ala region of the sacrum.

35. The method of claim 32, wherein the at least three anatomical structures includes a superior endplate, an anterior wall of a sacral body, and an alar slope of the sacrum.

36. The method of claim 32, wherein the inserting includes inserting the medical device such that the medical device does not enter the breach zone.

37. The method of claim 32, wherein the anatomical landmark is a pedicle of the sacrum.

38. The method of claim 32, wherein the anatomical landmark is a V notch of the sacrum.

39. A kit, comprising:
a cannula configured to be inserted into a sacral ala region of a sacrum; and
an expandable device configured to be inserted into the sacral ala region via a lumen of the cannula,
the expandable device having a distal end portion configured to be expanded from a collapsed configuration to an expanded configuration within the sacral ala region such that a cavity is formed within the sacral ala region.

40. The kit of claim 39, further comprising:
an access tool configured to provide an access path into a sacral ala region of a sacrum, the expandable device configured to be inserted into the sacral ala region of the sacrum via the access path.

41. The kit of claim 39, further comprising:
a delivery device movably disposable within the lumen of the cannula, the delivery device configured to inject bone cement into at least one of a lumen of the cannula or the cavity.

42. The kit of claim 39, further comprising:
an access tool having a sharp distal end configured to penetrate bone, the access tool configured to provide an access path into a sacral ala region of a sacrum,
the expandable device configured to be inserted into the sacral ala region of the sacrum via the access path.

43. The kit of claim 39, wherein the distal end portion of the expandable device is configured to form a low-pressure zone within the sacral ala region when expanded within the sacral ala region of the sacrum.

44. The kit of claim 39, wherein the distal end portion of the expandable device is configured to be expanded within the sacral ala region of the sacrum until the distal end portion of the expandable device contacts at least one of a cortical wall, an anterior ala, a superior ala, a sacral foramen or a sacroiliac joint of the sacrum.

45. The kit of claim 39, wherein the distal end portion of the expandable device is configured to be expanded within the sacral ala region of the sacrum until the distal end portion of the expandable device has an interior volume of at least approximately 3 cubic centimeters.

46. The kit of claim 39, wherein the distal end portion of the expandable device is configured to be expanded within the sacral ala region of the sacrum until the distal end portion of the expandable device has an interior pressure of at least approximately 400 psi.

47. The kit of claim 39, wherein the distal end portion of the expandable device is configured to be expanded within the sacral ala region of the sacrum in substantially 0.5 cubic centimeter increments.

48. The kit of claim 39, wherein the distal end portion of the expandable device includes a balloon.

49. The kit of claim 39, wherein the cannula is sized to be inserted percutaneously.

50. The kit of claim 39, wherein the cannula is configured to be inserted via a medial-to-lateral approach and based on a fluoroscopic image.
